(19) United States
(12) Patent Application Publication
(10) Pub. No.: US 2022/0062247 A1
(43) Pub. Date: Mar. 3, 2022

(54) USE OF A PAR-1 ANTAGONIST FOR THE TREATMENT OF A CHRONIC INFLAMMATORY INTESTINAL DISEASE

(71) Applicant: CVASTHERA, Labruguière (FR)

(72) Inventors: Bruno LE GRAND, Teyssode (FR); Sylvie SABLAYROLLES, Teyssode (FR)

(21) Appl. No.: 17/415,376

(22) PCT Filed: Dec. 18, 2019
(86) PCT No.: PCT/EP2019/086027

§ 371 Date: Jun. 17, 2021

(30) Foreign Application Priority Data
Dec. 19, 2018 (FR) 1873359

(51) Int. Cl. A61K 31/443; A61K 31/5377; A61K 31/495

(52) U.S. Cl. CPC A61K 31/443; A61P 29/00; A61K 31/495; A61K 31/5377

(57) ABSTRACT

Disclosed is the use of a PAR-1 antagonist, in particular selected from vorapaxar, atopaxar and 3-2-(chloro-phenyl)-1-[4-(4-fluoro-benzyl)-piperazine-1-yl]propenone, for the prevention and/or treatment of a chronic inflammatory disease of the intestine and of the colon in a mammal, in particular Crohn's disease.

### BACKGROUND OF THE INVENTION

### Field of the Invention

The present invention relates to the field of treatment of chronic bowel diseases.

More particularly, the present invention relates to a PAR-1 antagonist, and a pharmaceutical composition containing such an antagonist, for their use for the prevention and/or treatment of a chronic inflammatory disease of the intestine and of the colon, in particular to reduce the pain and/or repair the epithelial tissues of the intestine in a subject suffering from such a disease.

### Description of the Related Art

Chronic inflammatory diseases of the intestine and of the colon, also called inflammatory bowel diseases, commonly referred to by the abbreviation IBDs, comprise Crohn's disease and haemorrhagic rectocolitis.

Crohn's disease can affect the entirety of the digestive tract, either in contiguous sections or in isolated sections, but it first and foremost affects the small intestine and the colon. The inflammation can affect the inner mucosa and even cross the entire thickness of the intestinal wall, it is manifested by oedema, an expansion of blood vessels and a fluid loss in the tissues. Crohn's disease is a pathology of young adults which generally starts between 20 and 30 years. There is a second frequency peak between 50 and 80 years and 15% of the cases concern children. Both sexes are equally affected. The affection is ubiquitous but its incidence is higher in Northern Europe than in Southern Europe. In France, the incidence of Crohn's disease is from 5 to 6 cases for 100,000 inhabitants and as many haemorrhagic rectocolitis. Moreover, the prevalence exponentially increases in industrialising countries, such as Maghreb, Asian and South African countries, etc.

The origin of Crohn's disease is still unknown. It is probably multifactorial associating a genetic predisposition—a gene of predisposition to chronic inflammatory bowel diseases has recently been discovered in humans on the chromosomes 12 and 16—and environmental factors that are still to be elucidated, for example a triggering infection or pollution. Moreover, the harmful role of tobacco is clearly established, thereby increasing the risk of relapse.

The haemorrhagic rectocolitis, or ulcerative colitis, is a chronic inflammatory bowel disease that affects the distal end of the digestive tract, that is to say the colon and the rectum which is always affected. Its aetiology is unknown, although a genetic component is a hypothesis. It is classified as an autoimmune disease. This disease cannot be cured, which requires a lifelong medication. The objective of the treatments is to make remissions last as long as possible. Its diagnosis is essentially based on cytological examinations which accompany the taking of samples during a colonoscopy.

These chronic inflammatory diseases of the intestine and of the colon develop by inflammatory eruptions with extremely variable duration and frequency from one patient to another. These eruptions alternate with remission phases.

During the inflammatory eruptions, the IBDs are most often characterised by abdominal pains, frequent diarrhoeas, sometimes bloody, or an affection of the anal region (fissure, abscess). These symptoms confer some taboo on the disease. Often, they are accompanied with fatigue, anorexia and fever, or extra-intestinal (articular, cutaneous, ocular, hepatic) manifestations. In about 20% of patients, the attacks are severe: their intensity could impose hospitalisation, waiving food and an infusion treatment for a few days. Furthermore, the evolution of the disease may result in a narrowing of the affected intestinal segment, and then an occlusion or an abscess. This may lead to the formation of a fistula, that is to say opening of an abnormal communication route starting from the intestine towards another organ. These complications require a surgery. IBDs are associated to an increased risk of colorectal cancer, in particular when lesions are present at the colon.

The diagnosis of IBDs is based on several clinical, biological and medical imaging criteria. When clinical symptoms suggest an IBD, a biological check-up is carried out. It allows detecting an inflammatory syndrome, the presence of markers specific to IBDs, in particular the anti-Saccharomyces Cerevisiae antibodies (ASCA) and the anti-polynuclear neutrophil cytoplasmic antibodies (ANCA). A digestive endoscopy allows searching for the presence and location of lesions of the digestive tract, as well as taking samples.

Taking care of a patient suffering from an IBD involves many parameters related to the form of the disease and to the patient himself. Five categories of medicines are currently used in the basic treatment of IBDs. These are salicylates, corticoids, immunosuppressants, biotherapies and antibiotics.

Among salicylates, the proven and the oldest one is represented by sulfasalazine. The risk of undesirable effects associated to this medicine has led to looking for better tolerated derivatives. These are mainly mesalazine and olsalazine.

An almost compulsory waypoint at some point of the evolution of the disease, the corticosteroid therapy has a bad reputation. It is reason for concern regarding the occurrence of many unpleasant effects. The treatment usually begins at high doses, and then the posologies are progressively reduced. In any case, corticoids require a medical monitoring.

Medicines that reduce the reactions of the immune system, or immunosuppressants, are increasingly used for the treatment of IBDs. They are the basis of the maintenance treatment of Crohn's disease, more rarely of the maintenance treatment of rectocolitis. They have an onset of action of a few months. The use of immunosuppressants imposes a regular monitoring of some blood parameters.

Biotherapies consist of the therapeutic use of products derived from the substances present in the living organism. The first medicine of this class used for the treatment of IBDs has been infliximab, it is the only product currently authorised by the health authorities. It consists of a chimeric antibody directed against the TNF-alpha. It is administered by short-term infusion. The side effects are dominated by the infections and the hypersensitivity reactions.

Finally, antibiotics are an adjunctive treatment of IBDs. Two of them occupy a prominent place in taking care of the anorectal occurrences of Crohn's disease: these are metronidazole and ciprofloxacin. However, both compounds are not devoid of side effects.

Schematically, a distinction is made between the treatment of the eruption aiming to bring the digestive tract to rest as quickly as possible, and the maintenance treatment aiming to maintain this remission as long as possible.

At present, there is no curative treatment for IBDs. Current anti-inflammatory medicines often allow for a durable control of the disease, even though they are not perfect. They prevent the apparition of eruptions and prolong the remission phases by promoting the cicatrisation of the lesions of the digestive tract.

In the absence of an effective remedy, patients suffering from Crohn's disease require continuous medical cares.

Furthermore, the absorption surface of the intestinal epithelium leaves a huge possibility of passage of foreign and toxic molecules into the blood and into the lymph. The disruption of this mechanical barrier and the passage of these substances into the systemic circulation, epithelial hyperpermeability, are the starting point common to many pathologies, and in particular chronic inflammatory diseases such as Crohn's disease and the haemorrhagic rectocolitis. Currently, there is no treatment that protects from this translocation of the epithelial barrier and/or accelerates the process of cicatrisation of the lesioned epitheliums, the current treatments of the chronic inflammatory diseases of the intestine aiming to reduce the inflammatory eruption without repairing the substrate.

### SUMMARY OF THE INVENTION

The present invention aims to provide new compounds allowing fighting IBDs in an effective way.

The publication of Vergnolle et al., 2004, Journal of Clinical Investigation, 114(10): 1444-1456 describes the role of the PAR-1 receptor in the inflammatory diseases of the intestine. This document indicates that a particular PAR-1 antagonist, the L-arininamide-4-methoxy-N-[[[1-[92,6-dichlorophenyl-methyl]-3-(1-pyrrolidinylmethyl)-1H-indol-6-yl]amino]carbonyl]-1-phenylalanyl-N-(phenylmethyl)-(5), allows improving the survival rate of animals in an induced colitis model in mice, by reducing the inflammation phenomenon.

It has now been discovered by the present inventors that, entirely surprisingly, particular PAR-1 receptor antagonists not only allow reducing the inflammatory phenomenon involved in chronic inflammatory diseases of the intestine and of the colon, and in particular Crohn's disease, but they also allow reducing the pain associated thereto as well as repairing the epithelial tissues of the intestine.

The PAR-1 (standing for protease-activated receptor-1) receptor is a trimeric G protein-coupled hepta-helical receptor, composed by 425 amino acids. Thrombin activates the PAR-1 receptor by cleaving its extracellular N-terminal end between the arginine 41 and the serine 42. The cleaved peptide has no particular activity, the new N-terminal end of the receptor serves as an agonist by folding towards the cellular surface and by interacting with the extracellular domains.

PAR-1 plays a key role in platelet activation at low thrombin concentrations, such that its role has been established in the field of vascular biology and atherothrombosis. PAR-1 antagonists have emerged as new promising and orally active antithrombotics. In this respect, mention may be made of vorapaxar and atopaxar which have provided promising clinical data (Capodanno et al., 2012, J. Thromb. Haemost. 10(10): 2006-15). Besides, vorapaxar has been granted FDA's registration in 2014 for the indication “reduction of thrombotic events in patients having a myocardial infarction or peripheral arterial diseases history”. The use of 3-2-(chloro-phenyl)-1-[4-(4-fluoro-benzyl)-piperazine-1-yl]propenone, another PAR-1 antagonist, in the cardio-vascular field, in particular as a platelet aggregation inhibitor, in both curative and preventive treatments of arterial or venous thrombosis, of stable angina, of cardiac rhythm disorders, of myocardial infarction, of hypertension, of heart failure, of cerebrovascular accidents, of acute coronary syndromes, has also been described in the prior art, illustrated in particular by the document WO 2007/147824.

The expression of the PAR, and in particular PAR-1, receptors in the digestive tract is described in the literature, in particular in the publication of Vergnolle, 2004, in J. Pharmacol, 141, 1264-1274.

However, nothing in the works described in this publication, or in the prior art in general, in particular in the aforementioned publication of Vergnolle et al., 2004, Journal of Clinical Investigation, 114(10): 1444-1456; suggested a role of antagonists that are specific to this receptor with respect to the pain and the damages at the intestinal epithelium involved in the context of these diseases, like how the present inventors have now discovered.

Thus, according to a first aspect, the present invention relates to a PAR-1 antagonist selected in the group constituted by vorapaxar, vorapaxar isomers having an antagonist activity with respect to the PAR-1 receptor, atopaxar, 3-2-(chloro-phenyl)-1-[4-(4-fluoro-benzyl)-piperazine-1-yl]propenone and their pharmaceutically acceptable salts, for its use for the prevention and/or treatment of a chronic inflammatory disease of the intestine and of the colon in a subject, and in particular for its use to relieve the pain and/or repair the epithelial tissues of the intestine in said subject suffering from a chronic inflammatory disease of the intestine and of the colon.

In particular, this subject is a mammal, for example a non-human mammal. Preferably, it consists of a human being.

By treatment, it is meant in the present description a curative treatment of the disease, and more particularly, the reduction and/or the inhibition of the development of at least one of the symptoms of the disease, the increase of the remission phase and/or the reduction of the number of attacks or of their frequency. In particular, the PAR-1 antagonist according to the present invention allows, inter alia, relieving the pain symptom related to the disease in an effective way.

By prevention, it is meant understood the fact of reducing, and even completely avoiding, the apparition of the disease.

By “repair the epithelial tissues”, it is meant, conventionally to a person skilled in the art, the fact of reducing the intestinal epithelial hyperpermeability and of re-establishing, at least partially, the mechanics of the intestine.

By antagonist of the PAR-1 receptor (also called PAR-1 antagonist), it is meant in the present description, in a manner conventional per se, a compound that interacts with the PAR-1 receptor, of the species of the considered subject, and opposes the effect of its natural ligand, thrombin, or by proteases. In general, the PAR-1 antagonists act by direct interaction with the PAR-1 receptor to block activation thereof.

In particular, the present invention relates to a PAR-1 antagonist for its use for the treatment of chronic inflammatory diseases of the intestine and of the colon in a subject in need thereof, that is to say suffering from one or more of these diseases, and more particularly to reduce the pain and/or repair the epithelial tissues of the intestine in this subject, said PAR-1 antagonist being administered to said subject in a therapeutically effective amount.

The chronic inflammatory disease of the intestine and of the colon targeted by the present invention may be Crohn's disease or the haemorrhagic rectocolitis.

Vorapaxar, atopaxar and 3-2-(chloro-phenyl)-1-[4-(4-fluoro-benzyl)-piperazine-1-yl]propenone are known PAR-1 antagonists, as it has been indicated hereinbefore. Their therapeutic use allows preventing and treating IBDs in a particularly effective manner, which has never been described or suggested in the prior art.

The vorapaxar, also called ethyl N-[(1R,3aR,4aR,6R,8aR,9S,9aS)-9-[(E)-2-[5-(3-fluorophenyl)pyridin-2-yl]ethenyl]-1-methyl-3-oxo-3a,4,4a,5,6,7,8,8a,9,9a-decahydro-1H-benzo[f][2]benzofuran-6-yl]carbamate (SCH-530348, CAS No. 618385-01-6), has the formula (I):

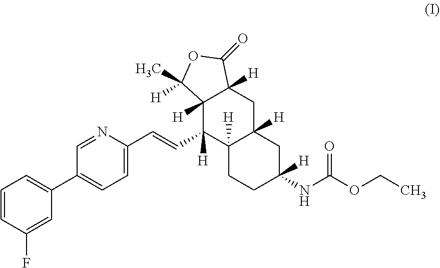

This compound is described in particular in the document WO 03/089428. In general, it is in the form of a sulphate salt.

By vorapaxar isomer, it should be understood any molecule having the same chemical formula as vorapaxar and having any possible combination of isomer forms at its asymmetrical carbons, that is different from that of vorapaxar. Starting from a mixture of isomers, each particular isomer may be obtained by purification methods that are conventional per se for the person skilled in the art. For example, an isomer of vorapaxar having an antagonist activity with respect to the PAR-1 receptor is the compound SCH 530348, described in the publication of Chackalamannil et al., 2008, J. Med. Chem. 51: 3061-3064.

Determining among all possible isomers of vorapaxar which ones have an antagonist activity with respect to the PAR-1 receptor is within the skills of the person skilled in the art. To this end, the person skilled in the art can in particular carry out protein binding tests against the PAR-1 agonist thrombin, as described in the aforementioned publication of Chackalamannil et al., 2008.

The atopaxar, or 1-(3-tert-butyl-4-methoxy-5-morpholin-4-ylphenyl)-2-(5,6-diethoxy-4-fluoro-3-imino-1H-isoindol-2-yl)ethanone (E5555, SCH-602539, CAS No. 751475-53-3) has the formula (II):

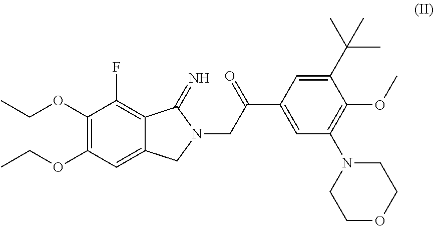 [SMILES: CCOc1cc2c(c(F)c1OCC)C(=N)N(CC(=O)c1cc(N3CCOCC3)c(OC)c(C(C)(C)C)c1)C2]

3-2-(chloro-phenyl)-1-[4-(4-fluoro-benzyl)-piperazine-1-yl]propenone has the formula (III):

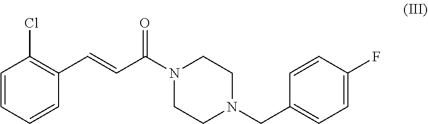 [SMILES: O=C(/C=C/c1ccccc1Cl)N1CCN(Cc2ccc(F)cc2)CC1]

By pharmaceutically acceptable salt, it is meant in the present description any salt of said compounds having, as a counterion, a species that produces no adverse, allergic effect or other undesirable reactions when it is administered to a subject, in particular to a mammal.

Any non-toxic conventional salt of the PAR-1 antagonists according to the invention, whether formed from organic or inorganic acids, could be used according to the invention. As example, mention may be made of the salts derived from inorganic acids such as the hydrochloric, hydrobromic, phosphoric, sulphuric acids, and the salts derived from organic acids such as the acetic, trifluoroacetic, propionic, succinic, fumaric, malic, tartaric, citric, ascorbic, maleic, glutamic, benzoic, salicylic, toluenesulfonic, methanesulfonic, stearic, lactic acids, etc.

These salts may be synthesised from a PAR-1 antagonist according to the invention and the corresponding acids, according to any chemical method that is conventional per se.

The PAR-1 antagonist may be used as such, or in the form of a solvate, for example in water or ethanol.

Another aspect of the invention relates to a pharmaceutical composition containing a PAR-1 antagonist as an active substance, and at least one pharmaceutically acceptable excipient, the PAR-1 antagonist being selected in the group constituted by vorapaxar, vorapaxar isomers having an antagonist activity with respect to the PAR-1 receptor, atopaxar, 3-2-(chloro-phenyl)-1-[4-(4-fluoro-benzyl)-piperazine-1-yl]propenone and their pharmaceutically acceptable salts, for its use, as a medicine, for the prevention and/or treatment of a chronic inflammatory disease of the intestine and of the colon, and in particular of Crohn's disease, in a subject, and more particularly for its use to relieve the pain and/or repair the epithelial tissues of the intestine in said subject suffering from a chronic inflammatory disease of the intestine and of the colon.

In particular, this subject may be a mammal, for example a non-human mammal, or preferably a human being.

Besides the PAR-1 antagonist, the pharmaceutical composition according to the invention may contain any pharmaceutically acceptable excipient.

Pharmaceutically acceptable herein means that the excipient has no adverse, allergic effect or other undesirable reactions when it is administered to a mammal, in particular to a human.

Such an excipient may be a diluent, an adjuvant or any other substance that is conventional per se for the constitution of medicines, such as a preservative, filler, disintegrating, wetting, emulsifying, dispersing, antibacterial or antifungal agent, or an agent allowing delaying the intestinal and digestive absorption and resorption, etc., or any one of the mixtures thereof.

The pharmaceutical composition according to the invention may further contain one or several other active substance(s), acting or not in synergy with the PAR-1 antagonist according to the invention, for example a pain-relieving substance.

The pharmaceutical composition according to the invention may be formulated according to any dosage form, in particular a form suited for an administration in mammals, and in particular in human beings.

The pharmaceutical composition according to the invention may be administered to the subject by oral, sublingual, subcutaneous, intramuscular, intravenous, transdermal, local or rectal route. In this case, the active substance may be administered in unitary forms, mixed with conventional pharmaceutical carriers. Suitable unitary forms comprise forms for oral administration such as pills, capsules, powders, granules and oral solutions or suspensions, forms for sublingual and buccal administration, forms for subcutaneous or transdermal, topical, intramuscular, intravenous, intranasal or intraocular administration, and forms for intravesical, intramural or rectal administration.

Preferably, the pharmaceutical composition according to the invention is orally administered to the subject. It then has a dosage form suited for such an oral administration. This dosage form may be conventional per se.

The pharmaceutical composition according to the invention may be solid and for example in the form of pills, capsules or granules.

In particular, pills may be obtained by mixing the active substance with a pharmaceutically compatible vehicle such as gelatin, starch, lactose, magnesium stearate, talc, gum arabic, silica or the same. The pills may be coated with sucrose or other suitable materials or be treated such that they have a prolonged or delayed activity and that they continuously release, preferably in the intestine and the colon, a predetermined amount of the active substance.

Otherwise, the pharmaceutical composition according to the invention may be in the form of capsules, obtained by mixing the active substance with a diluent and incorporating the obtained mixture into soft or hard capsules.

Powders and granules, in particular water dispersible, may be formed by mixing the active substance with dispersing agents, wetting agents and/or suspension agents, as well as with taste-masking agents or sweeteners.

Alternatively, the pharmaceutical composition according to the invention may be in a fluid form, in particular in the form of a syrup or an elixir containing the active ingredient together with a sweetener, an antiseptic, as well as a flavouring agent and an adequate colourant.

Otherwise, the pharmaceutical composition according to the invention may be administered to the subject by rectal route. It then has a suited dosage form, for example of a gel, a cream, a powder, a suspension, a solution, a foam or a suppository, which are prepared with binders melting at rectal temperature, for example cocoa butter or polyethylene glycols.

Otherwise, the pharmaceutical composition according to the invention may be administered to the subject by parenteral (intravenous, intramuscular, intradermal, subcutaneous), intranasal, intraocular, intravesical or intramural route. It then has a suited dosage form, for example of an aqueous suspension, an isotonic saline solution or a sterile and injectable solution, which contains pharmaceutically compatible dispersing agents and/or wetting agents.

Otherwise, the active substance may be formulated in the form of microcapsules, possibly with one or several additive carrier(s).

The adequate formulations for each selected particular administration form are known to the person skilled in the art and described, for example, in the work of Remington, The Science and Practice of Pharmacy, 19^{th} edition, 1995, Mack Publishing Company.

Preferably, the concentration of the PAR-1 antagonist in the pharmaceutical composition according to the invention is selected so as to dispense to the subject, at each administration, an amount of this antagonist that is effective to obtain the desired therapeutic response, in particular the desired level of reduction of the pain and/or level of repair of the epithelial tissues of the intestine. The therapeutically effective dose level of the PAR-1 antagonist specific for a particular subject varies according to many factors such as the actual pathology and its seriousness, the chosen administration route, the body weight, the age, the gender and the overall health condition of the subject, the duration of the treatment, the medicines possibly used in parallel, the sensitivity of the individual to be treated, etc.

Consequently, the optimum posology should be determined by the specialist in the matter according to the parameters he considers relevant.

Although the effective doses can vary to a large extent, the daily doses of the active substance according to the invention could range between 0.1 mg and 1000 mg every 24 hours, preferably between 1 and 100 mg every 24 hours and preferably between 1 and 10 mg every 24 hours, in one or several dose(s), preferably in one single dose.

Preferably, the administration of the pharmaceutical composition according to the invention begins the soonest as of the diagnosis of the disease and, preferably, within the 12 first months following the acute event.

The present invention can also be expressed in terms of a method for preventing and/or treating a chronic inflammatory disease of the intestine and of the colon, in particular Crohn's disease, in a subject, and in particular a method for relieving the pain and/or repairing the epithelial tissues of the intestine in a subject suffering from a chronic inflammatory disease of the intestine and of the colon. In particular, the subject may be a mammal and preferably a human being. This method comprises the administration to said subject of a therapeutically effective amount of a PAR-1 antagonist as defined hereinbefore, in particular vorapaxar or one of its pharmaceutically acceptable salts, or of a pharmaceutical composition containing a PAR-1 antagonist as defined hereinbefore and at least one pharmaceutically acceptable excipient.

This method may have one or several of the feature(s) described hereinbefore with reference to the use of the PAR-1 antagonist and/or of the pharmaceutical composition containing it.

In the embodiments of the invention wherein the pharmaceutical composition according to the invention, containing a PAR-1 antagonist as defined hereinbefore as an active substance and at least one pharmaceutically acceptable excipient, has a solid dosage form suited for an oral administration, the PAR-1 antagonist may be included in a core covered with an enteric coating.

By enteric, it is meant in the present description, in a manner conventional per se, the fact that this coating prevents any release of the active substance before having reached the intestine and the colon, thereby preventing in particular any direct contact between the active substance and the gastric mucosa. Thus the enteric coating is not degraded in the upper portion of the intestinal tract, which has a high acidity, and it enables a release of the active substance essentially in the distal ileum and the colon, where the intestinal pH progressively increases up to values ranging from 5 to 7.

For example, the pharmaceutical composition according to the invention may be in the form of a capsule containing a homogeneous dispersion of the active substance in a hydrophilic matrix, for example comprising a cellulose derivative, the whole forming a core that is covered with a coating for example based on ethyl cellulose or based on a polymer derived from acrylic acid and methacrylic acid esters, such as the polymers commercialised under the commercial name Eudragit®.

For example, such a capsule may contain between 1 and 10 mg, for example about 2.5 mg, of the PAR-1 antagonist. For example, it may be administered orally, at the rate of one capsule a day.

Examples of dosage forms in accordance with the invention, for an oral administration and aiming an enteric release of the active substance, are described in particular in the documents WO 2009/114773, US 2015/0196518 or WO 2005/193788, the teaching of these documents being transposable to the PAR-1 antagonist according to the invention.

The present invention is also expressed in terms of use of a PAR-1 antagonist selected in the group constituted by vorapaxar, vorapaxar isomers having an antagonist activity with respect to the PAR-1 receptor, atopaxar, 3-2-(chloro-phenyl)-1-[4-(4-fluoro-benzyl)-piperazine-1-yl]propenone and their pharmaceutically acceptable salts, for the manufacture of a medicine for the treatment and/or the prevention of a chronic inflammatory disease of the intestine and of the colon, and in particular to reduce the pain and/or repair the epithelial tissues of the intestine in a subject suffering from a chronic inflammatory disease of the intestine and of the colon, in particular from Crohn's disease.

This use may meet one or more of the feature(s) described hereinbefore with reference to the PAR-1 antagonist and to the pharmaceutical composition according to the invention.

### BRIEF DESCRIPTION OF THE DRAWINGS

The features and advantages of the invention will appear more clearly in light of the examples of implementation hereinafter, provided merely for an illustrative purpose and without limiting the invention, based on FIGS. 1 to 12, wherein:

FIG. 1 shows a graph illustrating the evolution over time of the body weight (Δweight) of rats in which a colitis has been induced by TNBS, after the colitis induction, for untreated rats (“TNBS”), for rats treated on a daily basis with prednisolone (“TNBS+Pred”) and for rats treated on a daily basis with vorapaxar (“TNBS+VX”), the control representing untreated rats in which no colitis has been induced;

FIG. 2 shows a graph illustrating the evolution over time of the body weight (Δweight) of rats in which a colitis has been induced by TNBS, after the colitis induction, for untreated rats (“TNBS”), for rats treated on a daily basis with prednisolone (“TNBS+Pred”) and for rats treated on a daily basis with the compound according to the invention CSI (“TNBS+CSI”), the CSI being administered at doses of 10 or of 40 mg/kg/day, the control representing untreated rats in which no colitis has been induced;

FIG. 3 shows a graph illustrating the evolution over time of the DAI score of rats in which a colitis has been induced by TNBS, after the colitis induction, for untreated rats (“TNBS”), for rats treated on a daily basis with prednisolone (“TNBS+Pred”) and for rats treated on a daily basis with vorapaxar (“TNBS+VX”), the control representing untreated rats in which no colitis has been induced;

FIG. 4 shows a graph illustrating the evolution over time of the DAI score of rats in which a colitis has been induced by TNBS, after the colitis induction, for untreated rats (“TNBS”) and for rats treated on a daily basis with the compound according to the invention CSI (“TNBS+CSI”), the CSI being administered at doses of 10 or 40 mg/kg/day, the control representing untreated rats in which no colitis has been induced;

FIG. 5 shows a graph illustrating the thickness of the wall of the colon of rats in which a colitis has been induced by TNBS, 7 days after the colitis induction, for untreated rats (“TNBS”), for rats treated on a daily basis with prednisolone (“TNBS+Pred”) and for rats treated on a daily basis with vorapaxar (“TNBS+VX”), the control representing untreated rats in which no colitis has been induced;

FIG. 6 shows a graph illustrating the macroscopic damage score in rats in which a colitis has been induced by TNBS, 7 days after the colitis induction, for untreated rats (“TNBS”), for rats treated on a daily basis with prednisolone (“TNBS+Pred”) and for rats treated on a daily basis with vorapaxar (“TNBS+VX”), the control representing untreated rats in which no colitis has been induced;

FIG. 7 shows a graph illustrating the macroscopic damage score in rats in which a colitis has been induced by TNBS, 7 days after the colitis induction, for untreated rats (“TNBS”), for rats treated on a daily basis with prednisolone (“TNBS+Pred”) and for rats treated on a daily basis with the compound according to the invention CSI (“TNBS+CSI”), the CSI being administered at doses of 10 or 40 mg/kg/day, the control representing untreated rats in which no colitis has been induced;

FIG. 8 shows a graph illustrating the myeloperoxidase (MPO) activity in rats in which a colitis has been induced by TNBS, 7 days after the colitis induction, for untreated rats (“TNBS”), for rats treated on a daily basis with prednisolone (“TNBS+Pred”) and for rats treated on a daily basis with vorapaxar (“TNBS+VX”), the control representing untreated rats in which no colitis has been induced;

FIG. 9 shows a graph illustrating the myeloperoxidase (MPO) activity in rats in which a colitis has been induced by TNBS, 7 days after the colitis induction, for untreated rats (“TNBS”), for rats treated on a daily basis with prednisolone (“TNBS+Pred”) and for rats treated on a daily basis with the compound according to the invention CSI (“TNBS+CSI”), the CSI being administered at doses of 10 or 40 mg/kg/day, the control representing untreated rats in which no colitis has been induced;

FIG. 10 shows a graph illustrating the evolution of the nociceptive response induced by TNBS in rats, as a function of the Von Frey filaments mass, 7 days after the colitis induction, for untreated rats (“TNBS”) and for rats treated on a daily basis with prednisolone (“TNBS+Pred”), the control representing untreated rats in which no colitis has been induced;

FIG. 11 shows a graph illustrating the evolution of the nociceptive response induced by TNBS in rats, as a function of Von Frey filaments mass, 7 days after the colitis induction, for untreated rats (“TNBS”) and for rats treated on a daily basis with vorapaxar (“TNBS+VX”), the control representing untreated rats in which no colitis has been induced;

and FIG. 12 shows graphs representing the percentage of the lesioned surface for epithelial cells of human colon on which a lesion has been made, brought into contact with vorapaxar at different concentrations or with the vehicle alone (“Control”), respectively a/4 h after the lesion, b/22 h after the lesion and c/28 h after the lesion.

### DESCRIPTION OF THE PREFERRED EMBODIMENTS

### Example 1: Assessment of Potential Therapeutic Effects of PAR-1 Antagonists in a Model of an Inflammatory Bowel Disease in Rats

The purpose of this study is to assess the effectiveness of two PAR-1 antagonists, vorapaxar and 3-2-(chloro-phenyl)-1-[4-(4-fluoro-benzyl)-piperazine-1-yl]propenone (hereinafter, referred to as CSI) in a model of an inflammatory bowel disease induced by 2,4,6-trinitrobenzenesulfonic acid (TNBS) in a male laboratory rat of the Wistar strain. This model is described in the publication of Whittle et al., 2003, in Methods Mol. Biol., 225, 209-222.

In this induced colitis model, the inflammatory reaction is measured every day and is well established 7 days after intra-colonical administration of TNBS.

Different parameters are measured: the weight of the rats, the presence of blood in the faeces and the diarrhoeas assessed by the disease activity index (DAI score), the intensity of pain (assessed by Von Frey filaments technique measured on days 3 and 7).

The rats are sacrificed 7 days after the beginning of the treatment and the macroscopic damages, the oedema and a marker of the inflammation, myeloperoxidase (MPO), are quantified.

Protocol

The experiments with the PAR-1 antagonists are carried out in two studies, relating to vorapaxar and CSI, respectively.

The colitis is induced by intra-colonical administration of 2,4,6-trinitrobenzenesulfonic acid (TNBS) (30 mg of TNBS in 0.25 ml of a 50% ethanol/NaCl solution) via a catheter (polyethylene (PE)-60) inserted 8 cm proximally from the anus.

Four groups of 10 rats receive an intra-colonical administration of TNBS, as well as a per os treatment of vorapaxar at the dose of 2.5 mg/kg/day or of CSI at doses of 10 and 40 mg/kg/day or of prednisolone (a compound proposed by the prior art for the treatment of IBDs) at the dose of 3 mg/kg/day. The per os treatments start 1 h before the intra-colonical administration of TNBS.

As a negative comparative example, 1 group of 10 rats receives an intra-colonical administration of TNBS and of the vorapaxar vehicle (phosphate buffered saline—PBS) or of the CSI vehicle (carboxymethyl cellulose—CMC at 1%) per os daily.

Moreover, 1 group of 6 control rats receives an intra-colonical administration of NaCl instead of TNBS.

As regards the 1^{st} study, using vorapaxar, 4 groups of rats are treated as indicated in Table 1 hereinbelow.

| | | | | Number of |
|---|---|---|---|---|
| | Intra-colonical | Per os | Per os treatment | individuals of |
| Group | administration | treatment | dose/24 h | the group |
| 1 | NaCl | — | — | 6 |
| 2 | TNBS | PBS | — | 10 |
| 3 | TNBS | vorapaxar | 2.5 mg/kg | 10 |
| 4 | TNBS | prednisolone | 3 mg/kg | 10 |

As regards the 2^{nd} study, using the CSI compound, 5 groups of rats are treated as indicated in Table 2 hereinbelow.

| | | | | Number of |
|---|---|---|---|---|
| | Intra-colonical | Per os | Per os treatment | individuals |
| Group | administration | treatment | dose/24 h | of the group |
| 1 | NaCl | — | — | 6 |
| 2 | TNBS | CMC 1% | — | 10 |
| 3 | TNBS | CSl | 10 mg/kg | 10 |
| 4 | TNBS | CSl | 40 mg/kg | 10 |
| 5 | TNBS | prednisolone | 3 mg/kg | 10 |

The DAI score is determined as follows. The development of the disease is estimated on a daily basis. The consistence of the excrements, as well as the presence of blood in the faeces assessed with the Hemoccult® paper test, are respectively quantified by the seriousness scales of Table 3 hereinbelow. The obtained values are added together to obtain the DAI score.

| Excrement consistency scale | | Hemoccult ® score | |
|---|---|---|---|
| 0 | Normal | 0 | Normal |
| 0.5 | Slightly soft excrements | 0.5 | No visible blood and |
| | | | Hemoccult ® ± |
| 1 | Soft excrements | 1 | No visible blood and |
| | | | Hemoccult ® + |
| 1.5 | Low diarrhoea | 1.5 | No visible blood and |
| | | | Hemoccult ® ++ |
| 2 | Diarrhoea | 2 | Visible blood |

The macroscopic damage score is assessed according to the protocol described in the publication of Comera et al., 1999, Dig. Dis. Sci. 44: 1448-1457, as follows. On day 7 after induction of the colitis, the rats are sacrificed by carbon dioxide CO₂ inhalation, the colon (from the anus to the Caecum) is removed and the macroscopic damages are assessed according to the parameters observed and presented in Table 4 hereinbelow, to determine the macroscopic damage score.

| Characteristic | Score |
|---|---|
| Normal appearance | 0 |
| Hyperaemia, no ulcer | 1 |
| Ulceration with no hyperaemia or thickening of | 2 |
| the intestinal wall | |
| Ulceration with inflammation over 1 area | 3 |
| Ulceration/inflammation over 2 areas or more | 4 |
| Major damage areas extended by >1 cm on the | 5 |
| length of the colon | |
| Major damage areas extended by >2 cm on the length | 6-10 |
| of the colon - the score is increased by 1 for each | |
| additional damaged cm | |

Results

Significant and representative clinical signs appear in this model of colitis induced by TNBS, such as diarrhoeas and the presence of blood in the faeces (DAI) as of the beginning of the study. Afterwards, some daily chronicity is established, assessed by the different parameters hereinafter.

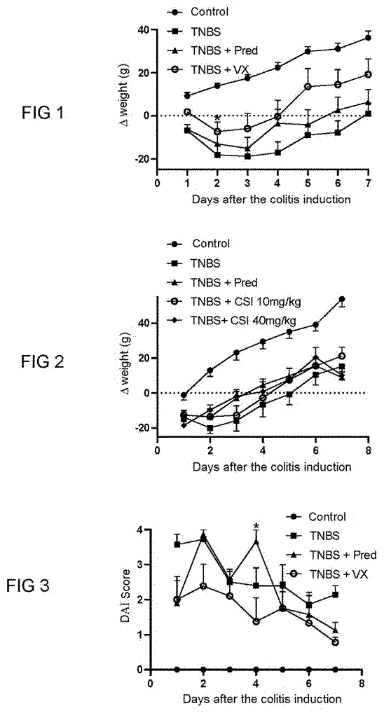

The evolution of the body weight of the rats over time is shown in FIG. 1 for vorapaxar and in FIG. 2 for CSI. These results show that a significant weight loss appears as of day 1 after the TNBS (“TNBS”) injection in comparison with the naive rats that have not undergone a colitis injection (“Control”) which have a daily weight gain. Vorapaxar significantly reduces this weight loss as of the 2^{nd} day. CSI, as well as prednisolone, alleviate this weight loss.

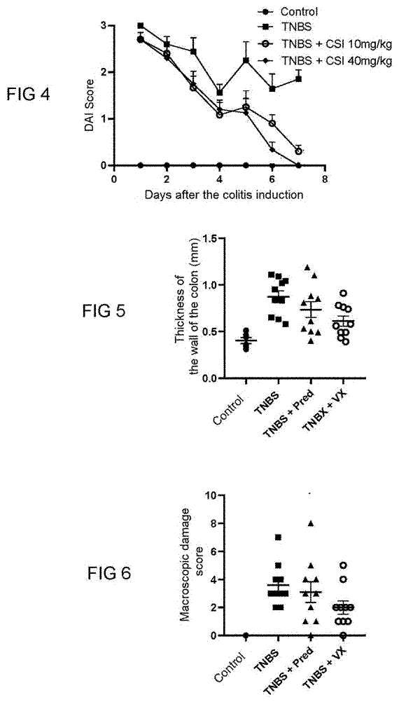

The evolution of the DAI score over time is shown in FIG. 3 for vorapaxar and in FIG. 4 for CSI.

The induction of colitis causes different severe clinical signs in rats such as diarrhoeas and the presence of blood in the faeces (DAI score) in the animals having been subjected to TNBS (“TNBS”) in comparison with the group of naïve rats (“Control”). This clearly shows that the administration of TNBS replicates all of the parameters of colitis.

The daily oral administration of prednisolone at the dose of 3 mg/kg does not prevent the increase of the disease activity index (DAI) in all observed points. This result proves that the rats subjected to TNBS are highly affected and that it is difficult to improve their symptoms.

Conversely, the daily administration of 2.5 mg/kg of vorapaxar or of 10 mg/kg of the compound CSI significantly reduces the disease activity index on the 7^{th} day. The disease activity index is significantly reduced as of the 1^{st} day of the treatment with vorapaxar at 2.5 mg/kg in comparison with the vehicle group (“TNBS”). These results clearly show that the PAR-1 antagonists according to the invention reduce the parameters of the colitis in this model.

Moreover, the colitis induction results in the apparition of oedema, which is quantified by measuring the macroscopic thickness of the tissue of the wall of the colon.

The effect of vorapaxar on the thickness of the tissues is shown in FIG. 5.

As it can be observed, the daily oral administration of prednisolone at the dose of 3 mg/kg does not prevent the apparition of oedema. Herein again, this result shows that the rats to which TNBS has been administered are highly affected and that it is difficult to improve their symptoms.

Conversely, the daily administration of vorapaxar at 2.5 mg/kg significantly reduces the apparition of this oedema on the 7^{th} day. Thus, the thickness of the wall of the colon that is representative of the apparition of the oedema in the intestinal tissue is significantly reduced with the treatment with vorapaxar at 2.5 mg/kg, in comparison with the vehicle group (“TNBS”).

The effects of the PAR-1 antagonists on the inflammatory parameters are assessed by determining the macroscopic damage score and by quantifying the activity of the myeloperoxidase (MPO) marker.

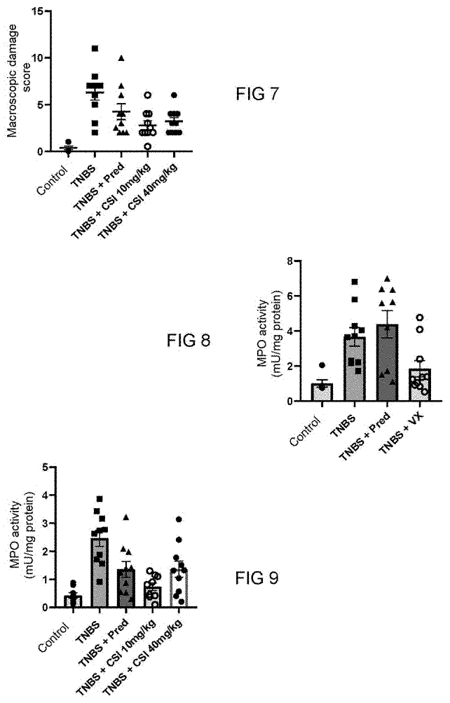

The effect on the macroscopic damage score is shown in FIG. 6 for vorapaxar and in FIG. 7 for CSI.

The effect on the MPO activity is shown in FIG. 8 for vorapaxar and in FIG. 9 for CSI.

Seven days after the beginning of the administration of TNBS (“TNBS”), the macroscopic damage score is significantly higher in comparison with that one obtained with the naïve rats (“Control”). A macroscopic examination of the colon of the rats subjected to TNBS shows that it has severe erythemas and the presence of oedemas and ulcers.

Myeloperoxidase (MPO), mainly expressed in granulocytes, is a marker of the inflammation. The presence of lesions of the mucosas, associated to a leukocyte infiltration, results in a rise of the MPO activity.

In comparison with the vehicle group (“TNBS”), the treatment with prednisolone at 3 mg/kg significantly reduces the macroscopic damage score; similarly, the macroscopic examination of the colons of these rats allows concluding that erythemas, the oedemas and the ulcers are less severe than those observed in the vehicle group (“TNBS”).

In comparison with the vehicle group (“TNBS”), the treatment with vorapaxar and with CSI at the tested doses significantly reduces the macroscopic damage score. The macroscopic examination of the colons also shows a significant reduction of erythemas, oedemas and ulcers.

Similarly, the treatments with the prednisolone and with the PAR-1 antagonists according to the invention, at the tested doses, significantly reduce the MPO activity in comparison with the vehicle group (“TNBS”). At the dose of 40 mg/kg/day of the compound CSI, it is observed that this activity is significantly reduced.

These results clearly show that the colitis induced by TNBS causes a significant increase of the inflammatory parameters (macroscopic damages and MPO activity), this increase being significantly reduced by the tested PAR-1 antagonists.

The intensity of the pain is measured by the Von Frey filaments technique on days 1, 3 and 7, according to the protocol described in the publication of Auge et al., 2013, Eur. J. Pharmacol. 707: 32-40.

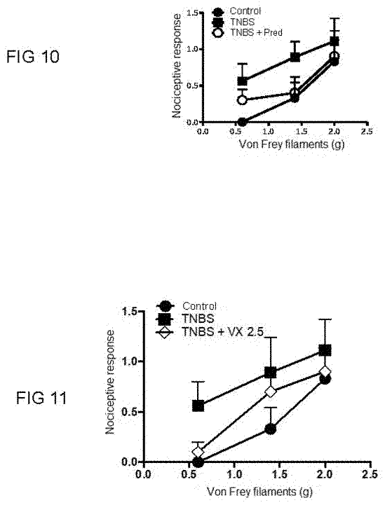

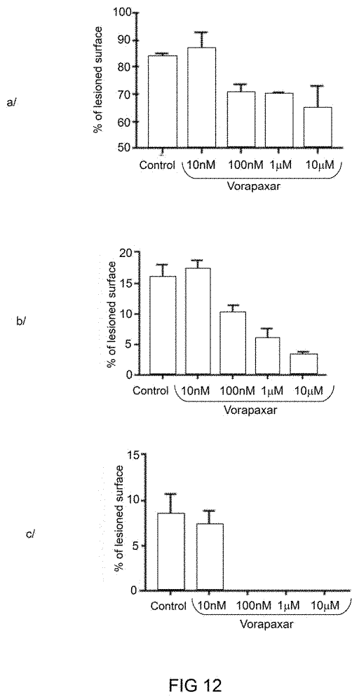

The effect on the nociceptive response, measured by Von Frey filaments technique according to the mass of Von Frey filaments, is shown in FIG. 10 for prednisolone and in FIG. 11 for vorapaxar.

It is observed that vorapaxar at the dose of 2.5 mg/kg/day significantly inhibits the allodynia-type response. In comparison, prednisolone induces only a partial inhibition.

These results prove that the colitis induced by TNBS causes a significant increase of the nociceptive response (allodynia), this increase being significantly reduced by vorapaxar.

### Example 2: Assessment of Potential Therapeutic Effects of a PAR-1 Antagonist in a Model of an Inflammatory Bowel Disease in Mice

The purpose of this study is to assess the effectiveness of a PAR-1 antagonist according to the invention, 3-2-(chloro-phenyl)-1-[4-(4-fluoro-benzyl)-piperazine-1-yl]propenone (CSI) in a model of an inflammatory bowel disease, Dextran Sulphate sodium (DSS) in mice. This DSS model is described in the publication of Choi et al., 2010, in J. Biomed. Biotechnol., 2010:943516; doi: 10.1155/2010/943516.

This model features a cytokine profile Th1 in its acute phase, periods of remission and relapses, it is consequently very similar to ulcerous colitis. The DSS distributed in the drinking water can cause an inflammation of the digestive tract and produce colorectal tumours in the rodent. The DSS is widely used as an animal model of human inflammatory diseases of the digestive tract.

The present study is conducted in 7-week-old mice (C57B16). The 5-aminosalicylic acid serves as a positive comparative example.

The first day of the study starts with the daily administration of a beverage containing DSS at 5% p/p in water to the mice, until the day of sacrifice. The development of the disease is estimated on a daily basis, with accurate weighting of the mice, the assessment of the consistence of the excrements as well as the presence of blood in the faeces with the Hemoccult® paper test. The DAI score is calculated as indicated in Example 1.

Each day, groups of mice receive per os CSI at different concentrations, or 5-aminosalicylic acid (5-ASA), or the vehicle alone, that is to say carboxymethyl cellulose (CMC) at 1%.

The mice are sacrificed 7 days after the beginning of the treatment. The colon is excised and the macroscopic damages are assessed. The colon is then cut into 4 pieces, the distal portion is placed in an Eppendorf tube and stored at −80° C. until the measurement of the activity of myeloperoxidase and the quantification of proteins. The proximal portion is kept in formaline at 10% for future histological analyses. The two other central portions are placed in Eppendorf tubes and stored at −80° C. for future analyses.

Protocol

The mice are distributed in 5 groups as indicated in Table 5 hereinbelow.

| | | | | Number of |
|---|---|---|---|---|
| | DSS | | Treatment dose/ | individuals |
| Group | administration | Treatment | 24 h | of the group |
| 1 | 0% | — | — | 8 |
| 2 | 5% | CSl | 10 mg/kg | 10 |
| 3 | 5% | CSl | 40 mg/kg | 10 |
| 4 | 5% | CMC alone | 100 μl | 10 |
| 5 | 5% | 5-ASA | 50 mg/kg | 10 |

Results

The variation of the body weight of the mice in comparison with day 0 is shown for each group in Table 6 hereinbelow. In this table, “Moy” refers to the average obtained for the different individuals of the group, and “esm” represents the standard deviation with respect to the average.

| | Variation of the body weight of the mice in comparison with day D0 (g) | | | | | | | |
|---|---|---|---|---|---|---|---|---|
| Group | Moy/esm | D1-D0 | D2-D0 | D3-D0 | D4-D0 | D5-D0 | D6-D0 | D7-D0 |
| 1 | Moy | 0.54 | 0.70 | 1.14 | 1.21 | 1.16 | 1.19 | 1.17 |
| | esm | 0.43 | 0.70 | 0.32 | 0.32 | 0.65 | 0.49 | 0.54 |
| 2 | Moy | 0.67 | 0.44 | 0.45 | −0.42 | −2.15 | −3.79 | −5.19 |
| | esm | 0.42 | 0.38 | 0.34 | 0.36 | 0.79 | 1.05 | 1.59 |
| 3 | Moy | −0.08 | −0.07 | −0.19 | −0.68 | −1.07 | −2.69 | −4.00 |
| | esm | 1.25 | 1.70 | 2.00 | 2.05 | 0.61 | 0.60 | 0.92 |
| 4 | Moy | 0.32 | 0.29 | 0.38 | −0.35 | −1.89 | −3.82 | −4.80 |
| | esm | 0.45 | 0.80 | 0.51 | 0.57 | 0.67 | 0.83 | 1.06 |
| 5 | Moy | 0.64 | 0.64 | 1.05 | −0.03 | −1.67 | −3.68 | −4.76 |
| | esm | 0.53 | 0.42 | 0.40 | 0.64 | 0.54 | 0.85 | 1.33 |

The evolution of the disease activity index of the mice over time is shown for each group in Table 7 hereinbelow. In this table, “Moy” refers to the average obtained for the different individuals of the group, and “esm” represents the standard deviation with respect to the average.

| | Disease activity index | | | | | | | |
|---|---|---|---|---|---|---|---|---|
| Group | Moy/esm | D1 | D2 | D3 | D4 | D5 | D6 | D7 |
| 1 | Moy | 0.00 | 0.00 | 0.00 | 0.00 | 0.00 | 0.00 | 0.00 |
| | esm | 0.00 | 0.00 | 0.00 | 0.00 | 0.00 | 0.00 | 0.00 |
| 2 | Moy | 1.40 | 1.35 | 2.00 | 3.75 | 4.00 | 4.00 | 2.90 |
| | esm | 0.55 | 0.47 | 0.76 | 0.50 | 0.00 | 0.00 | 0.57 |
| 3 | Moy | 2.00 | 1.10 | 1.50 | 2.80 | 4.00 | 4.00 | 3.00 |
| | esm | 1.00 | 0.55 | 0.50 | 1.10 | 0.00 | 0.00 | 0.71 |
| 4 | Moy | 1.57 | 1.50 | 2.60 | 3.83 | 4.00 | 4.00 | 3.90 |
| | esm | 0.45 | 0.50 | 0.55 | 0.41 | 0.00 | 0.00 | 0.32 |
| 5 | Moy | 1.64 | 1.25 | 1.92 | 3.50 | 4.00 | 4.00 | 3.20 |
| | esm | 0.75 | 0.52 | 0.20 | 0.58 | 0.00 | 0.00 | 0.79 |

The mice subjected to DSS (Group 4) develop colitises, which is demonstrated by a weight loss and the disease activity index which has significantly increased.

The weight loss of the mice subjected to DSS is present as of the 4^{th} day and is significant for days 5, 6 and 7 in comparison with the mice that have not been subjected to DSS (Group 1).

The disease activity index becomes very rapidly different in the mice subjected to DSS (Group 4) in comparison with the control mice (Group 1), as shown in Table 7.

All these results clearly show that the administration of DSS replicates all of the parameters of colitis.

The daily oral administration of 5-ASA at the dose of 50 mg/kg (Group 5) prevents neither the weight loss nor the increase of the disease activity index in all observed points. This result proves that the mice subjected to DSS are highly affected and that it is difficult to improve their symptoms.

Conversely, the daily administration of CSI at 10 mg/kg (Group 2), significantly reduces the disease activity index on the 7^{th} day. At the dose of 40 mg/kg (Group 3), the disease activity index is significantly reduced on the 4^{th} and on the 7^{th} day of the treatment in comparison with the vehicle group (Group 4). In addition, at this dose, the compound according to the invention CSI significantly reduces the weight loss on the 6^{th} day. These results demonstrate a protective effect of this compound in this colitis model in mice.

On day 7, after having sacrificed the animals, the colon is removed. The macroscopic damage score, assessed as described in Example 1, and the myeloperoxidase (MPO) activity are determined. The obtained results are summarised in Table 8 hereinbelow.

| | | Macroscopic damage | MPO activity |
|---|---|---|---|
| Group | Moy/esm | score | (mU/mg proteins) |
| 1 | Moy | 0.0 | 0.80 |
| | esm | 0.0 | 0.45 |
| 2 | Moy | 5.2 | 12.99 |
| | esm | 0.7 | 3.93 |
| 3 | Moy | 4.0 | 6.9 |
| | esm | 0.6 | 2.5 |
| 4 | Moy | 9.6 | 10.97 |
| | esm | 0.5 | 1.68 |
| 5 | Moy | 6.5 | 9.34 |
| | esm | 0.8 | 3.17 |

Seven days after the beginning of the treatment with DSS (Group 4), the macroscopic damage score is significantly higher in comparison with that obtained with the control mice (Group 1). A macroscopic examination of the colon of the mice subjected to DSS shows severe erythemas and the presence of oedemas and ulcers. The infiltration of inflammatory cells is estimated by measuring the MPO activity, a granulocytic infiltration index. The administration of DSS results in a very considerable and statistically significant increase of the MPO activity in comparison with the control mice.

In comparison with the vehicle group (Group 4), the treatment with 5-ASA at 50 mg/kg (Group 5) significantly reduces the macroscopic damage score; similarly, the macroscopic examination of the colons of these mice allows concluding that the erythemas, the oedemas and the ulcers are less severe than those observed in the vehicle group.

In comparison with the vehicle group (Group 4), the treatment with the compound CSI according to the invention at both tested doses (Groups 2 and 3) significantly reduces the macroscopic damage score. The macroscopic examination of the colons also shows a significant reduction of erythemas, oedemas and ulcers.

Furthermore, at the dose of 40 mg/kg of the compound CSI according to the invention (Group 3), the MPO activity is strongly reduced.

These results show that 3-2-(chloro-phenyl)-1-[4-(4-fluoro-benzyl)-piperazine-1-yl]propenone reduces, significantly and in a dose-dependent way, the daily signs of colitis, and seems to be more effective than 5-ASA.

The results presented hereinbefore in Example 1 and Example 2 clearly prove that the treatment with a PAR-1 antagonist, whether this consists of vorapaxar or 3-2-(chloro-phenyl)-1-[4-(4-fluoro-benzyl)-piperazine-1-yl]propenone, significantly reduces the inflammation and the pain in two models of induced Crohn's disease, respectively in rats and in mice.

### Example 3: Galenic Formulations

Examples of pharmaceutical compositions according to the invention, in a dosage form suited for an oral administration, for a release of the active substance essentially in the distal ileum and the colon, are described hereinafter.

Formula 1

The pharmaceutical composition according to the invention is in the form of a microgranules containing vorapaxar, atopaxar or 3-2-(chloro-phenyl)-1-[4-(4-fluoro-benzyl)-piperazine-1-yl]propenone, mixed with the following excipients: microcrystalline cellulose, magnesium stearate.

These microgranules are coated with a semi-permeable layer of ethyl cellulose which enables the diffusion of the active molecules present in the microgranules.

They are in the form of pills each containing an amount comprised between 1 and 10 mg of active substance and between 0.02 and 1.2 mg of ethyl cellulose.

Formula 2

The pharmaceutical composition according to the invention is in the form of pills containing a core including vorapaxar, atopaxar or 3-2-(chloro-phenyl)-1-[4-(4-fluoro-benzyl)-piperazine-1-yl]propenone, mixed with the following excipients: sodium carbonate, glycine, povidone, microcrystalline cellulose, silica, calcium stearate, titanium dioxide, iron oxide.

The core is coated with a copolymer of methacrylic acid Eudragit® L100 to allow for an enteric protection of this core, with a release starting from the duodenum.

Each pill contains an amount comprised between 1 and 10 mg of active substance and between 0.01 and 1.0 mg of Eudragit® L100.

Posology

For each of the formulas hereinabove, the pharmaceutical composition may be administered at the rate of 1 pill per day in 1 single dose.

Preferably, the treatment begins the soonest as of the diagnosis of the disease and, preferably, within the 12 first months following the acute event.

### Example 4: Epithelial Cells Repair Effect

A lesion is practiced using a sterilised forceps on human colon epithelial cells Caco2 in monolayer and at a confluence.

Afterwards, the cells are rinsed twice with sterilised foetal bovine serum and then placed in an adequate culture medium. In this medium, vorapaxar at different concentrations (10 nM, 100 nM, 1 μM, 10 μM) or the vehicle (control) are added in independent culture dishes.

The cicatrisation of the lesion is supervised and analysed using a confocal microscope for 28 h. The percentage of the lesioned surface of the lesion is determined for each tested condition, 4 h, 22 h and 28 h after the lesion. The obtained results are shown in FIG. 12, respectively in a/, b/ and c/.

In a/ in the figure, it appears that 4 h after the lesion, the cicatrisation is highly enhanced by vorapaxar at the concentrations of 100 nM, 1 μM and 10 μM. After 22 h, in b/ in the figure, it can be observed that vorapaxar amplifies the cicatrisation surface in a concentration-dependent way. Finally, in c/ in the figure, it could be observed that after 28 h, vorapaxar at the concentration from 100 nm to 10 μM has caused the complete cicatrisation of the epithelial lesion.

Quite probably, this cicatrisation property participates in the reduction of the thickness of the inflamed colon in the TNBS model in rats, shown in FIG. 5.

This result shows that, through an amplification of the cicatrisation process, vorapaxar is capable of repairing the epithelial wall of a lesioned intestine, as it might occur in the case of hyperpermeability which precedes the inflammatory flare in patients suffering from Crohn's disease of from ulcerative colitis with a pain recurrence.

1. A method of reducing the pain and/or repairing the epithelial tissues of the intestine in a subject suffering from a chronic inflammatory disease of the intestine and of the colon, comprising the administration to said subject of a therapeutically effective amount of a PAR-1 antagonist selected from the group consisting of vorapaxar, vorapaxar isomers having an antagonist activity with respect to the PAR-1 receptor, atopaxar, 3-2-(chloro-phenyl)-1-[4-(4-fluoro-benzyl)-piperazine-1-yl]propenone and their pharmaceutically acceptable salts.

2. The method of claim 1, wherein said subject is a mammal.

3. The method of claim 1, wherein said subject is suffering from Crohn's disease.

4. A method of reducing the pain and/or repairing the epithelial tissues of the intestine in a subject suffering from a chronic inflammatory disease of the intestine and of the colon, comprising the administration to said subject of a therapeutically effective amount of a pharmaceutical composition containing a PAR-1 antagonist as an active substance and at least one pharmaceutically acceptable excipient, said PAR-1 antagonist being selected from the group consisting of vorapaxar, vorapaxar isomers having an antagonist activity with respect to the PAR-1 receptor, atopaxar, 3-2-(chloro-phenyl)-1-[4-(4-fluoro-benzyl)-piperazine-1-yl]propenone and their pharmaceutically acceptable salts.

5. The method of claim 4, wherein said subject is a mammal.

6. The method of claim 4, wherein said subject is suffering from Crohn's disease.

7. The method of claim 4, wherein said pharmaceutical composition has a dosage form suited for an oral administration.

8. The method of claim 4, wherein said PAR-1 antagonist is included in a core covered with an enteric coating.

9. The method of claim 2, wherein said subject is a human being.

10. The method of claim 5, wherein said subject is a human being.

11. The method of claim 5, wherein said subject is suffering from Crohn's disease.

12. The method of claim 5, wherein said pharmaceutical composition has a dosage form suited for an oral administration.

13. The method of claim 6, wherein said pharmaceutical composition has a dosage form suited for an oral administration.

14. The method of claim 5, wherein said PAR-1 antagonist is included in a core covered with an enteric coating.

15. The method of claim 6, wherein said PAR-1 antagonist is included in a core covered with an enteric coating.

16. The method of claim 7, wherein said PAR-1 antagonist is included in a core covered with an enteric coating.

17. The method of claim 11, wherein said pharmaceutical composition has a dosage form suited for an oral administration.

18. The method of claim 11, wherein said PAR-1 antagonist is included in a core covered with an enteric coating.

19. The method of claim 12, wherein said PAR-1 antagonist is included in a core covered with an enteric coating.

20. The method of claim 13, wherein said PAR-1 antagonist is included in a core covered with an enteric coating.
